(19) United States
(12) Patent Application Publication
(10) Pub. No.: US 2026/0232861 A1
(43) Pub. Date: Aug. 13, 2026

(54) Lite Air Tecnologies DAC

(71) Applicant: Francis N. Welch Hernandez, SAN ANTONIO, TX (US)

(72) Inventor: Francis N. Welch Hernandez, SAN ANTONIO, TX (US)

(21) Appl. No.: 19/049,595

(22) Filed: Feb. 10, 2025

(51) Int. Cl. A61L 9/22; A61L 9/18; H02J 3/32; H02J 3/38

(52) U.S. Cl. CPC A61L 9/22; A61L 9/18; H02J 3/32; H02J 3/381; A61L 2209/111; A61L 2209/12; A61L 2209/14; H02J 2101/24

(57) ABSTRACT

LITE Air Technologies DAC is a self-sustaining, eco-friendly direct air capture and air purification system designed to reduce atmospheric carbon dioxide, airborne particulates, metals, bacteria, viruses, and mold. The unit incorporates a turbine-powered air intake, multi-layer filtration including carbon-capturing cloth, a fusion light chamber that sterilizes 99.99% of pathogens, and an electromagnetic module for extracting airborne metals. The structure includes integrated solar panels, battery backup, and a bidirectional inverter for grid interaction. The translucent casing, ranging from 200 to 815 feet, emits ambient light and is engineered for deployment in industrial, urban, and high-emission environments to support environmental resilience and carbon mitigation. Captured contaminants are collected and processed offsite for recycling or conversion into usable material.

### BACKGROUND OF THE INVENTION

### Field of the Invention

The present invention relates to environmental engineering and air purification systems, particularly a large-scale, self-sustaining system designed to remove carbon dioxide, airborne pollutants, metals, and biological contaminants from the atmosphere.

### Description of Related Art

While various forms of air purification systems exist, most are limited in scale or scope and do not incorporate a unified system for capturing carbon, removing metals, and sterilizing pathogens in the air. There remains a need for a comprehensive, standalone system capable of addressing multiple pollutants simultaneously while operating sustainably off-grid.

### SUMMARY OF THE INVENTION

The invention disclosed herein is a self-sustaining, eco-friendly, large-scale air purification system, named LITE Air Technologies DAC, designed to draw in ambient air and remove multiple forms of pollution including carbon dioxide, airborne particulates, bacteria, viruses, mold, and metal particles. It operates using a combination of vacuum turbine intake, carbon-absorbing cloth, HEPA and ionization-based air purification, electromagnetic metal extraction, and a fusion light chamber for biological sterilization. Power is supplied by an integrated solar panel system with battery storage, and excess energy can be fed back into the grid. The system is housed in a translucent, bulletproof structure that lights up the surrounding sky, providing both aesthetic and functional value. Collected pollutants are transported offsite for conversion into usable materials such as jet fuel or metal recycling.

### DETAILED DESCRIPTION OF THE INVENTION

The LITE Air Technologies DAC unit includes the following components:
A vacuum-powered turbine intake system designed to pull in large volumes of ambient air.
Carbon-capturing cloth positioned to absorb atmospheric CO2.
Multi-layer air filtration including HEPA, activated carbon, and ionization units.
A fusion light chamber that emits high-frequency energy to neutralize 99.99% of bacteria, viruses, and mold.
An electromagnetic filtration system with adjustable intensity to capture airborne metal particulates.
Solar panels mounted to the exterior with battery banks capable of off-grid operation and energy storage.
A bidirectional inverter system to feed excess electricity back to the power grid.
A translucent and bulletproof structural enclosure designed to emit light for both functionality and visibility.
A data control and monitoring system to track air quality, operational status, and pollutant levels in real-time.
A transportable offsite collection and recycling hub where captured carbon is converted to fuel and metals are sorted for recycling.

### INDUSTRIAL APPLICABILITY

This system is suitable for industrial, municipal, and remote environmental applications. It can be implemented near power plants, oil refineries, large cities, highways, airports, and other high-emission zones to dramatically reduce air pollution.

### ADVANTAGES OF THE INVENTION

Operates independently with renewable solar energy and battery backup
Removes a wide spectrum of pollutants in one unified system
Provides real-time air monitoring and data tracking
Capable of grid-tied energy feedback for resilience
Designed with both durability and visual appeal
Recycles pollutants into reusable resources

### 2. Specification of Invention

LITE Air Technologies DAC is an advanced, self-sustaining, eco-friendly air purification system designed to combat atmospheric pollution on a large scale. The device stands 850 feet tall with a bulletproof structure to ensure durability and resilience in various environments. It operates using solar panels as the primary energy source, supported by backup battery systems to ensure continuous functionality in all conditions.

Key features include:
Vacuum Technology: A turbine system that draws in polluted air from the atmosphere, optimizing air intake for maximum efficiency.
Carbon-Capturing Cloth Technology: This innovative fabric traps and stores carbon dioxide, significantly reducing atmospheric CO₂ levels.
Fusion Light Technology: Utilizes advanced light fusion to kill 99.99% of airborne bacteria and viruses, ensuring clean, sterile air output without harming the ozone layer.
Electromagnetic System with On/Off Switch: Designed to attract and collect metal particles from the air. The system can be activated or deactivated as needed.
Recycling Process: The captured carbon is recycled for sustainable purposes, including conversion into jet fuel or general industrial recycling. Collected metals are also processed for reuse.
Self-Sustainability: Powered by solar panels with backup battery systems, ensuring 24/7 operation even during power outages or low sunlight conditions.
Eco-Friendly Design: Engineered with environmentally safe materials and processes to minimize ecological impact while promoting clean energy solutions.

This combination of technologies positions LITE Air Technologies DAC as a revolutionary solution for urban air purification, especially beneficial near industrial zones, oil refineries, and heavily polluted areas.

1. An eco-friendly, self-sustaining direct air capture and purification system comprising:
a) a multi-stage filtration assembly including:
i. a carbon-capturing cloth system for absorbing atmospheric carbon dioxide,
ii. a high-efficiency air purification unit configured to remove airborne particulates,
iii. a fusion light sterilization chamber capable of eliminating at least 99.99% of airborne bacteria and viruses,
iv. an electromagnetic filtration module operable to extract airborne metallic particles;
b) a solar panel array coupled with a backup battery storage system, configured to operate independently of external power sources and feed surplus energy back into the grid to enhance energy resilience;
c) a vacuum turbine system configured to draw ambient air into the filtration assembly;
d) a translucent structural housing designed to emit visible light and illuminate the surrounding atmosphere;
e) an offsite processing facility for recycling captured carbon and metal particles into usable materials.

2. The system of claim 1, wherein the solar power system is integrated with a bidirectional inverter to enable grid-tied energy feedback during periods of surplus generation.

3. The system of claim 1, wherein the structural housing uses eco-friendly, recycled, or low-impact materials and is designed for minimal land disruption.

4. The system of claim 1, wherein the air purification unit comprises HEPA, activated carbon, and ionization filters for enhanced particulate removal.

5. The system of claim 1, wherein the electromagnetic filtration module automatically activates based on airborne metal concentration detected by onboard sensors.

6. The system of claim 1, wherein the carbon-capturing cloth is regularly removed and transported to an offsite facility for processing into fuel or sequestered materials.

7. The system of claim 1, further comprising a central control unit configured to monitor air quality, adjust system operations in real-time, and report environmental impact data to a cloud-based server.

8. The system of claim 1, wherein the translucent structural housing ranges from approximately 200 to 815 feet in height, comprises a transparent casing, and is designed to remove carbon pollution from elevated atmospheric levels, thereby enhancing large-scale air purification impact.
